(19) United States
(12) Patent Application Publication
(10) Pub. No.: US 2005/0096515 A1
(43) Pub. Date: May 5, 2005

(54) Three-dimensional surface image guided adaptive therapy system

(76) Inventor: Z. Jason Geng, Rockville, MD (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 10/973,579

(22) Filed: Oct. 25, 2004

Related U.S. Application Data
(60) Provisional application No. 60/514,142, filed on Oct. 23, 2003.

(51) Int. Cl. A61B 5/00; G03B 17/24

(52) U.S. Cl. 600/315; 396/310

(57) ABSTRACT

A patient surface image guided therapy process includes the steps of acquiring a three-dimensional reference image of an area to be treated, acquiring a three-dimensional treatment image of the area to be treated; matching the reference image to the treatment image; and calculating any differences between the reference image and the treatment images to generate patient repositioning parameters.

### RELATED APPLICATIONS

The present application claims priority under 35 U.S.C. § 119(e) from the following previously-filed Provisional Patent Application, U.S. Application No. 60/514,142, filed Oct. 23, 2003 by Geng, entitled “Novel 3D Surface Image Guide Adaptive Therapy System for Cancer Treatment” which is incorporated herein by reference in its entirety

### BACKGROUND

Stereotactic radiosurgery (SRS) has gained its popularity in treatments of small brain lesions. The SRS technique uses 3D image data from CT and/or MRI scans and dedicated treatment planning tools to guide multiple photon beams from either cobalt sources in a gamma knife unit or an x-ray source in a Linear Accelerator to deliver a single large dose to an intracranial tumor while sparing neighboring nerves. Clinical results from many institutions in the last two decades have demonstrated that the SRS can achieve the same tumor control but with no surgical invasion as compared with the traditional surgical resection.

In recent years, more investigators are interested in using fractionated stereotactic radiotherapy (FSR) as an alternative to SRS for management of the primary brain tumors and brain metastases. In contrast to the single large dose used in SRS, the FSR involves multiple treatment sessions to deliver a high biological equivalent dose to the tumor but much less biological equivalent doses to the neighboring nerves and critical structures with application of the specific dose-time pattern. Clinical results suggest that FSR could further improve the treatment for brain tumors.

One major issue remaining in using FSR over SRS is the increment of patient-head refixation in the daily treatments. In SRS, the head was fixed to the head-ring through screwing pins to the skull, and the head-ring could be rigidly fixed to the treatment machine. The uncertainty for the head refixation is about 0.5-mm. In contrast, the head refixation in FSR frequently uses a thermoplastic head holder that can be attached to the treatment machine in daily patient setup. A typical FSR head holder includes a posterior piece, a facemask, and a mouth-nose or upper jaw holder. By comparing orthogonal portal images with corresponding digital reconstructed radiographs (DRR), we have found that the patient's head can be displaced inside the facemask by up to 5-mm. The standard deviation in the longitudinal direction is about 2-mm, which is considerably large for a stereotactic-type treatment.

Current commercial systems for patient-head position verification are adopted for the technique of mapping light-fields on the positioning box, which verifies the patient support devices but not the patient's head inside the head holder. The head could be slightly rotated at repositioning within the thermoplastic head holder, causing significant error in head refixation. Recent efforts have been directed to two-dimensional image-guided position verification by mapping the daily portal images with CT-based digital-reconstructed radiographs. However, the radiograph-based patient-head position verification requires a large-field irradiation that can increase the dose to the radiosensitive critical structures.

Accurate refixation may also be relevant for the treatment of other types of cancer, such as breast cancer. One in every 8 American women develops breast cancer at some point in their lifetime. Approximately 4% of American women die of breast cancer. It is estimated that more than 250,000 new cases of breast cancer occur among American women each year. Breast Conserving Therapy (BCT), defined as excision of the primary tumor and adjacent breast tissue, followed by radiation therapy of the breast and/or regional lymph nodes, has been widely accepted as a treatment option for most women with clinical Stage I or II invasive breast cancer. Traditionally, for patients undergoing BCT, megavoltage radiation therapy is recommended to the whole breast using medial and lateral tangential fields treating to a dose of 45 to 50 Gy (1.8 to 2.0 Gy per fraction) over a 4½ to 5½ week period. This is usually followed by a boost of radiation therapy to the area of the excisional biopsy for an additional 10 to 20 Gy. The treatment technique is called whole-breast irradiation (WBI).

However, it is unclear if the entire breast needs to be treated, or only a more limited volume surrounding the tumor (Recht 2000). Evidence suggests that WBI is unnecessary for patients with certain histological and clinical factors (Solin et al 1986,Schnitt et al 1987,Holland et al 1990,Ngai et al 1991,Schnitt et al 1992,Morimoto et al 1993,Recht et al 1995,Recht et al 2000). Interstitial implantation of the breast with radioactive sources has been explored to irradiate a quadrant of the breast, and results indicate that treating only the area adjacent to the primary tumor may be as effective as WBI for certain patients with early-stage breast cancer (Ribeiro et all 990,Fentiman et al 1991,Ribeiro et al 1993,Vicini et al 1997,Vicini et al 1999,King et al 2000,Vicini et al 2001).

Irradiating a quadrant of the breast is a viable alternative to WBI. In WBI, a portion of the lung and chest wall, and sometimes the heart (when treating the left breast, as shown in FIG. 2), can receive a radiation dose as high as the dose in the target. The contralateral breast also receives some dose from the scattered radiation. The advantage of quadrant irradiation is that unnecessary irradiation to the heart, chest wall, lung, and the contralateral breast can be significantly reduced because the target area is smaller. Thus the long-term complications such as cardiac damage and radiation pneumonitis may be reduced using quadrant irradiation (Pierce et al 1992,Shapiro et al 1994, 2001). Additionally, quadrant irradiation permits re-irradiation if the patient develops a new primary tumor in the same breast (Recht et al 2000).

Due to reduced toxicities, quadrant irradiation is able to adopt much higher fractional doses (e.g., 4 Gy per fraction BID), therefore significantly shortening the treatment time and potentially reducing health care costs (Vicini et al 2001). The course of treatment requires eight visits in four days as compared to the 30 needed during six weeks of WBI. The shorter treatment scheme makes quadrant irradiation more flexible for integration with chemotherapy, and more importantly, more convenient for the patient.

Because of the lengthy treatment course (6-7 weeks) required for the traditional WBI, many breast cancer patients who receive breast conserving surgery still do not receive adjuvant radiation therapy, despite strong evidence indicating improved outcomes with the addition of radiotherapy after breast conserving surgery. The greatly shortened treatment course for quadrant irradiation makes radiotherapy more appealing, particularly for patients who do not have easy access to a radiation oncology clinic. Accordingly, quadrant radiation may increase the number of women receiving the standard of care for their breast cancer treatment.

Quadrant irradiation can be realized through either the interstitial implantation of the breast with radioactive sources (called brachytherapy) or the clever use of megavoltage external beams (partial breast irradiation (PBI)). One disadvantage of brachytherapy is its difficulty. Brachytherapy requires considerable expertise for good results, and not all radiation oncologists perform brachytherapy procedures routinely enough to maintain surgical skills. Currently, only about a dozen or so institutions perform interstitial brachytherapy on a regular basis because it is so difficult to do and hard to teach. Brachytherapy requires operating room time and anesthesia, with added costs and possible side effects. Large volume implants may result in undesired high dose regions that cause fat necrosis. In addition, brachytherapy is invasive compared to PBI; many patients have problems with the idea of having needles or catheters temporarily placed in their breast.

The major technical challenge for a successful PBI treatment is the precise delivery of radiation dose to the subsurface target volume. Due to the mobility and potential deformation of breast tissue, it is difficult to precisely replicate the planned breast position on a daily basis. Respiration may also cause target motion during the treatment; however, the effect during quiet respiration is secondary to the daily variation. Breast motion and deformation is not a problem for brachytherapy and also not critical for WBI where the entire breast is contained by two tangential fields with adequate field margins. However, in PBI a high fractional dose (4 Gy/fraction) is supposed to be delivered to a small volume inside the breast. To spare as much normal tissue as possible, a small safety margin around the target volume is used. In such scenarios inaccurate localization of the target volume could result in PBI treatment failure due to
1) the local recurrence caused by geometric miss of the tumor, and
2) the unacceptable toxicity caused by irradiating normal tissue to high fractional and daily dose.

Therefore, precise targeting may be desirable for PBI. Precise targeting of internal breast lesions is technically challenging. In conventional WBI treatments, the patient is set up to the treatment position by matching skin markers to the wall/ceiling mounted lasers. Uncertainties in conventional laser-based setups are not negligible and are definitely unacceptable for PBI. X-ray imaging is not suitable for breast setup due to its poor quality for visualizing soft tissue. Opto-electronic systems using passive markers have been tested for breast cancer patient setup (Baroni et al 2000). However, the surface information provided by a finite set of markers placed on the patient skin is limited.

### SUMMARY

A 3D patient surface image guided therapy process includes the steps of capturing a 3D surface image of an area to be treated, preparing a pre-treatment CT scan of the area to be treated, matching the CT scan and the 3D surface image of the area to be treated, calculating any differences between the CT scan data and the 3D surface area images to generate patient repositioning parameters, and adjusting patient positioning or treatment machine configuration to achieve correct patient positioning.

### BRIEF DESCRIPTION OF THE DRAWINGS

The accompanying drawings illustrate various embodiments of the present apparatus and method and are a part of the specification. The illustrated embodiments are merely examples of the present apparatus and method and do not limit the scope of the disclosure.

FIG. 1 is a flowchart of a method for obtaining information about an area to be treated according to one exemplary embodiment.

FIG. 2A is schematic view of fiducial points according to one exemplary embodiment.

FIG. 2B is a schematic view of fiducial points according to one exemplary embodiment.

FIG. 2C is a flowchart of an iterative fine alignment optimization process according to one exemplary embodiment.

FIG. 3 is a schematic of a system for providing image guided therapy according to one exemplary embodiment.

FIG. 4 is a flowchart of an image guided adaptive therapy process according to one exemplary embodiment.

FIG. 5 illustrates a guidance correction interface according to one exemplary embodiment.

FIG. 6 illustrates a digital micro-mirror device.

FIG. 7 illustrates a rainbow projector according to one exemplary embodiment.

FIG. 8 illustrates a calibration fixture according to one exemplary embodiment.

Throughout the drawings, identical reference numbers designate similar, but not necessarily identical, elements.

### DETAILED DESCRIPTION

A method and system are provided herein for surface image guided therapy techniques. According to one exemplary embodiment, patient surface images are acquired using a three-dimensional camera when the patient is at the CT-simulation position and after setup for fractionated stereotactic treatment. The simulation and treatment images are aligned through an initial registration using several feature points followed by a refined automatic matching process using an iterative-closest-point mapping-align algorithm.

The video-surface images could be automatically transformed to the machine coordinate according to the calibration file obtained from a template image. Phantom tests have demonstrated that we can capture surface images of patients in a second with spatial resolution of submillimeter. A millimeter shift and one-degree rotation relative to the treatment machine can be accurately detected. The entire process takes about two minutes.

A method according to one exemplary embodiment includes patient repositioning and error correction based on accurate registration between the pre-operative CT scan and the 3D surface profiles of a patient's breast acquired during the treatment. Since 3D surface images can be acquired in real-time and will cause no additional irradiation, the re-positioning approach provides an elegant way to provide accurate and fast patient repositioning.

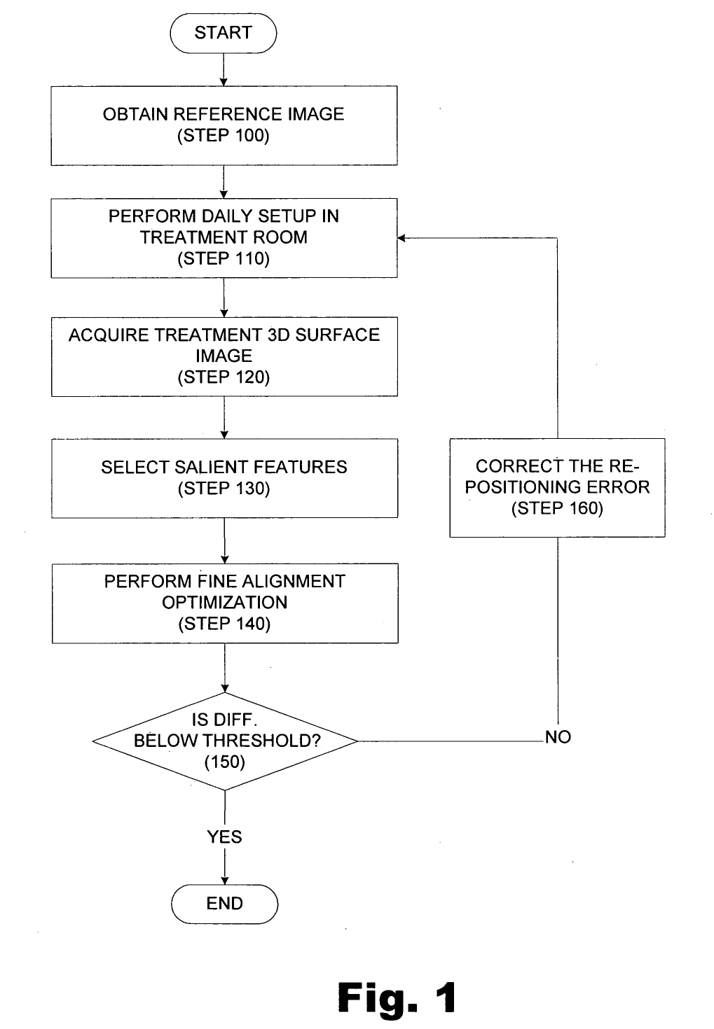

A generalized flowchart of one exemplary method is shown in FIG. 1. The method includes obtaining a reference image (step 100) such as CT scans or other suitable scans. Acquiring reference images may also include acquiring three-dimensional images. The combination of CT scans and three-dimensional surface images may provide detailed volumetric information. After the reference image has been obtained, a daily setup is performed in the treatment room (step 110). The present method may allow for more rapid and accurate treatments for patients.

Once the patient is positioned in the treatment room, a three-dimensional surface treatment image is acquired (step 120). The images are matched by selecting salient features (step 130) and then performing a fine alignment optimization routing (step 140). The difference between the reference image and the treatment image is calculated (step 150). If the difference between the position corresponding to the reference position and the position of the patient is not below a predetermined threshold (NO, 150) a refixation value is calculated (step 160) and the operator refixes or repositions the patient relative to the therapy machine and/or the camera. This process continues until the difference is below the threshold (YES, 150).

Accordingly, the present method provides surface image guided refixation or repositioning and non-invasive imaging such that harm due to radiation used in taking three-dimensional surface images may be reduced or eliminated. Further, the method and system may provide sub-millimeter measurement accuracy in images that are acquired in less than one second and registered in less than a minute. Each of these steps and the system used to capture and process the images will be discussed in more detail below.

3D Surface Image Based Positioning Error Detection and Correction Algorithms.

The repositioning error has been traditionally treated as a random error because the error cannot be detected. With help of the 3D-video imaging technique, the patient setup error in the real treatment session may be determined so that the “random error” can be unfolded and corrected. Several concepts of the position error are relevant to correction. First, there are initial setup errors, patient movement (position changes) between irradiation of different beams or arcs, and potential patient motion during the irradiation (when the beam is on). The initial setup error is measured by automatically aligning the patient surface image taken after the setup to the planned reference surface image. Multiple coplanar beams and arcs are routinely used in SRT, which involve table, gantry, and collimator rotations. From clinical experience, the table rotation is the major source of error causing the patient position changes (1-2 mm) between beams or arcs, thus position changes between irradiation of the beams/arcs have to be detected and corrected.

With a ceiling mounted 3D camera, the surface image may be instantly captured when the table is rotated to a new position. By mapping the new treatment surface images to the initial setup surface images, the relative shift and rotation of the head to the initial position can be determined. By subtracting the desired table rotation from the measured changes, the head position changes relative to the treatment machine can be determined. Further, such a configuration also allows the system to monitor the position of the imaged area during the radiation and make a quick interruption of the beam (arc) if significant (>1 mm) patient motion is detected from the 3D images. With this surface image guided patient refixation, all possible displacement of the isocenter and the rotations around the isocenter have been quantified and corrected according to the real-time images. Thus, the system may ensure accurate dose delivery through the entire course of treatment.

Accurate image registration between the 3D surface images acquired during the treatment and the reference 3D scan may be desirable to provide meaningful re-positioning information. The first step is to find corresponding points and the second step is to estimate the pose transformation from the point pairs. The 3D surface images S for patients are 3-tuples, i.e. S=(V, E, F) where V is vertex set, E is edge set and F is face set. Given two 3D surfaces, including a reference surface SR, and ST, a treatment surface, which are acquired with the patient in the CT simulation position and with the patient at each treatment respectively, our task is to align SR and ST, and further estimate patient's repositioning parameters.

Thus, according to one exemplary embodiment, the salient features are selected by an operator, such as by clicking a mouse. Once two sets of 3D surface images are loaded into the software, an operator can easily identify salient feature points, such as corners of eyes and mouth, from two surface images, via mouse clicking. To compensate for potential error of manual operation of not being able to click on the exact feature points, a refinement algorithm based on a correlation matching technique may be used to refine the locations of these corresponding points. The final outcome of the three pairs of feature points is then used for the image alignment algorithm.

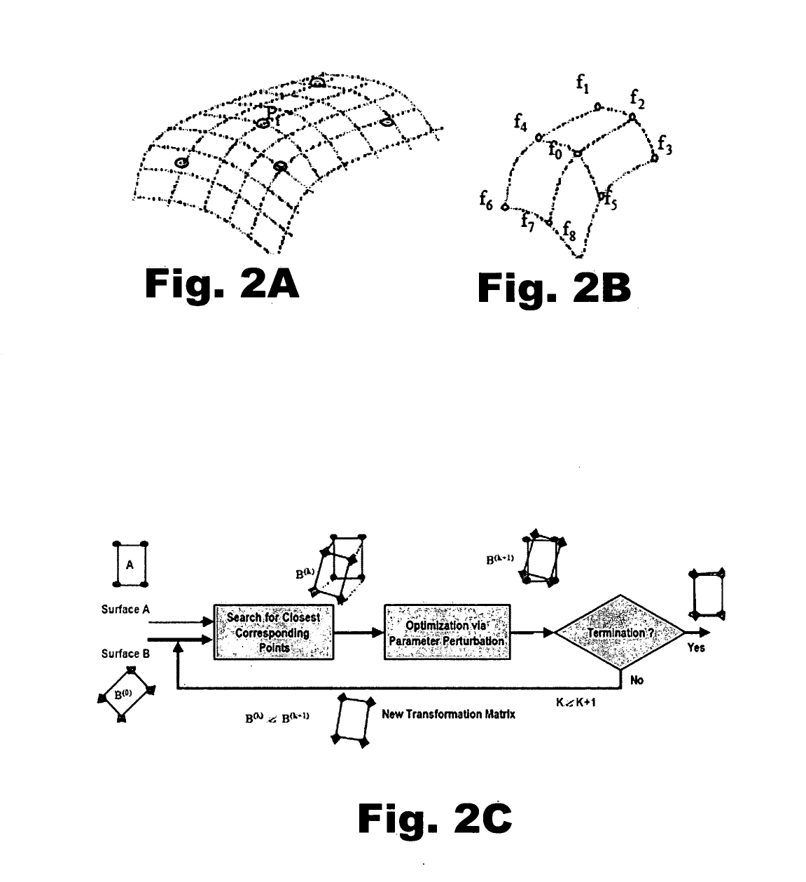

According to another exemplary embodiment, a set of features are selected, either automatically or by selection, by a set of fiducial points (such as approximately 50-100 3D surface points), which are assigned based on distinctive features (such as surface curvature) of the 3D facial surface profile (shown in FIG. 2A as Pi).

Around each of these fiducial points, we will extract the local surface characteristics ([x, y, z] coordinate value, surface curvatures, surface normal vector, etc) using a 3D data set of the neighboring points, as shown in FIG. 2B. The collection of the local features of all fiducial points forms a “feature vector” of this particular surface in this configuration. Instead of comparing all 3D surface data of a captured 3D image with that of the reference image, the feature vectors are compared to improve the processing speed and allow for the real-time 3D image comparison.

Geometric information of a 3D surface image can be represented by a triplet I=(x, y, z). To align a pair of 3D surface images, a set of salient fiducial points (i.e., local 3D landmarks) on one image is selected, and 3D features are defined for these points that are independent from the selection of 3D coordinate system. The objective of an automatic alignment algorithm is to automatically locate corresponding fiducial points from other 3D image and generate a transformation matrix that can convert the 3D image pair into a common coordinate system.

The local minimum curvature and maximum curvature is selected as the local feature vector whose value is determined by the geometric feature of the 3D surface, not by the selection of the coordinate system. At the location of each fiducial point a local feature vector is produced. A 3×3 window for a fiducial point f₀=(x_{0,y}₀,z₀) is defined, which contains all of its 8-connected neighbors {f_{w}=(x_{w},y_{w},z_{w}), w=1, . . . ,8}, as shown in FIG. 2A. A local feature vector is defined for the fiducial point as (k₀₁,k₀₂₎^{t}, where k₀₁ and k₀₂ is the minimum and maximum curvature of the 3D surface at the fiducial point, respectively. The details on the computation of the k₀₁ and k₀₂ follows:

Assume that the surface near the fiducial point can be characterized by:
z(x,y)=β₂₀x²+β₁₁xy+β₀₂y²+β₁₀x+β₀₁y+β₀₀. 1)

Consider the second order surface characterization for the fiducial point at f₀ and its 8-connected neighbors. The 3D surface at each of the 9 points in a ×'3 window centered on as one row in the following matrix expression may be expressed as:

$$[ \begin{matrix}{z}_{0} \\ {z}_{1} \\ {z}_{2} \\ {z}_{3} \\ {z}_{4} \\ {z}_{5} \\ {z}_{6} \\ {z}_{7} \\ {z}_{8}\end{matrix} ] = [ \begin{matrix}{x}_{0}^{2} & & {x}_{0} {y}_{0} & & {y}_{0}^{2} & & {x}_{0} & & {y}_{0} & & 1 \\ {x}_{2}^{1} & & {x}_{1} {y}_{1} & & {y}_{1}^{2} & & {x}_{1} & & {y}_{1} & & 1 \\ {x}_{2}^{2} & & {x}_{2} {y}_{2} & & {y}_{2}^{2} & & {x}_{2} & & {y}_{2} & & 1 \\ {x}_{3}^{2} & & {x}_{3} {y}_{3} & & {y}_{3}^{2} & & {x}_{3} & & {y}_{3} & & 1 \\ {x}_{4}^{2} & & {x}_{4} {y}_{4} & & {y}_{4}^{2} & & {x}_{4} & & {y}_{4} & & 1 \\ {x}_{5}^{2} & & {x}_{5} {y}_{5} & & {y}_{5}^{2} & & {x}_{5} & & {y}_{5} & & 1 \\ {x}_{6}^{2} & & {x}_{6} {y}_{6} & & {y}_{6}^{2} & & {x}_{6} & & {y}_{6} & & 1 \\ {x}_{7}^{2} & & {x}_{7} {y}_{7} & & {y}_{7}^{2} & & {x}_{7} & & {y}_{7} & & 1 \\ {x}_{8}^{2} & & {x}_{8} {y}_{8} & & {y}_{8}^{2} & & {x}_{8} & & y8 & & 1\end{matrix} ] [ \begin{matrix}{β}_{20} \\ {β}_{11} \\ {β}_{02} \\ {β}_{10} \\ {β}_{01} \\ {β}_{00}\end{matrix} ] ,$$

or Z =Xβ in vector form, where β=[β₂₀ β₁₁ β₀₂ β₁₀ β₀₁ β₀₀]^{t} is the unknown parameter vector to be estimated. Using the least mean square (LMS) estimation formulation, we can express β in terms of Z, X,
β≈\hat{β}=(X^{t}X)⁻¹X^{t}Z, 1)
where (X^{t}X)⁻¹X^{t} is the pseudo inverse for X. The estimated parameter vector \hat{β} is used for the calculations of the curvatures k₁ and k₂. Based on the definitions in differential geometry, k₁ and k₂ are computed based on the intermediate variables, E, F, G, e,f, g.
G=1+β₀₂ ² f=(2β₁₁)/\sqrt{EG−F²},
e=(2β₂₀)/\sqrt{EG−F²}, g=(2β₀₂)/\sqrt{EG−F²}.
The minimum curvature at the point f₀ is defined as:
k₁=[gE−2Ff+Ge−\sqrt{(gE+Ge−2Ff)²−4(eg−f²)(EG−F²)}]/[2(EG−F²)],
and the maximum curvature is defined as:
k₂=[gE−2Ff+Ge+\sqrt{(gE+Ge−2Ff)²−4(eg−f²)(EG−F²)}]/[2(EG−F²)].
where k₁ and k₂ are two coordinate-independent parameters indicating the minimum and the maximum curvatures at f₀, and forming the feature vector that represents local characteristics of the 3D surface.

As discussed in previous sections, the index function is defined as

$$I = {\sum }_{i = 1}^{n} {w}_{i} {\vert {A}_{i} - R ( {B}_{i} - {B}_{c} ) - t \vert}^{2} ,$$

where R is the function of three rotation angles and t is a translation vector such that (x,y,z), and Aᵢ and Bᵢ are the n corresponding sample points on surface A and B, respectively. The transformation matrix can be calculated using a three feature point pair. Given feature points A₁, A₂, and A₃ on surface A and corresponding B₁, B₂, and B₃ on surface B, a transformation matrix can be obtained by the following procedure:
1. Align B₁ with A₁ (via a simple translation);
2. Align B₂ with A₂ (via a simple rotation around A₁); and
3. Align B₃ with A₃ (via a simple rotation around A₁A₂ axis).

The combination of these three simple transformations will produce a transformation matrix. In the case where multiple feature points are available, we would examine all possible pairs (Aᵢ, Aⱼ, Aₖ) and (Bᵢ, Bⱼ, Bₖ), where i, j, k,=1,2, . . . N. We would rank the transformation matrices according to an error index

$$( I = {\sum }_{i = 1}^{n} {w}_{i} {\vert {A}_{i} - R ( {B}_{i} - {B}_{c} ) - t \vert}^{2} ) .$$

The transformation matrix that produces the minimum error will be selected.

Once the reference image and the treatment image have been coarsely aligned, the images are aligned using a fine feature process. Instead of using just the selected feature points, a large number of sample points Aᵢ and Bᵢ are used in the shared region, and the error index value for a given set of R and T parameters is calculated. Small perturbations to the parameter vector are generated in all possible first order differences, which results in a set of new index values. If the minimal value of this set of indices is smaller than the initial index value of this iteration, the new parameter set is updated and a new round of optimization begins. FIG. 2C shows the iterative fine alignment optimization process. Two sets of 3D images, denoted as surface A and surface B, are received or input. An initial guess is made of the transformation matrix (R⁽⁰⁾,t⁽⁰⁾) with initial parameter vector. A set of transformation (R′,t′) iteratively aligns A and B. Search Closest Point (250) is performed for any given sample point Aᵢ^{(k)} on surface A to find the closest corresponding Bᵢ^{(k)} on surface B, such that distance d=|Aᵢ^{(k)}−Bᵢ^{(k)}| is minimal for all neighboring points of Bᵢ^{(k)}. This step also includes calculation of an error index:

$${I}^{( k )} = {\sum }_{i = 1}^{n} {w}_{i} {\vert {A}_{i} - {R}^{( k )} ( {B}_{i}^{( k )} - {B}_{c} ) - {t}_{i}^{( k )} \vert}^{2}$$

Once the error index has been calculated, the error index for perturbed parameter vectors (αₖ±Δα,βₖ±Δβ,γₖ±Δγ,xₖ±Δx,yₖ±Δy,zₖ±Δz), is calculated where (Δα,Δβ,Δγ,Δx,Δy,Δz) are pre-set parameters. Thereafter, Compare Index Values of Perturbed Parameters and Decide an Optimal Direction (260) is performed. If the minimal value of this set of indices is smaller than the initial index value of this iteration k (NO, 270), the new parameter set is updated and a new round of optimization begins. Terminate: If the minimal value of this set of indices is greater than the initial index value of this iteration k (YES, 270), terminate the optimization process. The convergence of the iterative fine alignment algorithm can be easily proven. Notice that the following equation holds I^{(k+1)}≦I^{(k)}, k=1,2, . . . . Accordingly, the optimization process does not diverge.

Positioning Error Detection and Correction Procedures

A basic algorithm for 3D positioning error detection and correction is discussed below. In the simulator planning session, after a patient's position is verified by other image modality (such as radiographic images), a reference 3D image of the patient is acquired in the ideal treatment position, a selected set of fiducial points on the reference 3D image are calculated and the feature vector is defined, and a spatial relationship is defined among them to obtain a reference coordinate.

During the repositioning procedure (step 160; FIG. 1), after the operator properly places the patient to the treatment position similar to the original setup position, a new 3D image is acquired. Beginning with the first fiducial point, the corresponding point on new 3D image is searched. Once the first corresponding point on the new 3D image is found, the spatial relationship of the fiducial point is used to determine the possible locations of other fiducial points on the new 3D image. Local feature vectors of corresponding fiducial points on the reference image and the new 3D image are compared to find a rigid 4×4 homogenous transformation to minimize the weighted least-squared distance between pairs of fiducial points. The 4×4 homogenous transformation matrix will provide sufficient information to guide the operator to make the possible position correction.

Accordingly, acquired 3D surface images may be compared with the reference 3D surface image to generate quantitative parameters regarding the patient's positioning error in all six degrees-of-freedom, facilitating the re-position adjustment. Because the 3D surface image is acquired instantly, this frame-less patient repositioning system also provides a solution for the real-time detection and correction of patient motion relative to the treatment machine in a single fraction. Further, the present video alignment approach may allow for more precise alignment accuracy (up to 0.1 mm). Thus, the surface fitting method may achieve precise fitting due to the accuracy that can be achieved by the 3D camera.

In addition, the present system and method may reduce Human Operator Error. In particular, the automatic 3D alignment system described herein may reduce the possibility of random positioning errors associated with human operators to reproduce the same position day after day.

The system and method also provide Real-Time Re-adjustment. For example, the 3D camera based repositioning approach may have the capability of performing real-time repositioning to compensate the patient movement during the treatment in a non-invasive manner.

Coordinate Transformation: 3D Camera to Treatment Machine

A simple and accurate coordinate transformation of image from the video coordinate system, s-uvw, to the treatment machine coordinate system, o-xyz, may be determined by the equations of

$$( \begin{matrix}x \\ y \\ z \\ 1\end{matrix} ) = [ \begin{matrix}{R}_{11} & {R}_{12} & {R}_{13} & {t}_{x} \\ {R}_{21} & {R}_{22} & {R}_{23} & {t}_{y} \\ {R}_{31} & {R}_{32} & {R}_{33} & {t}_{z} \\ 0 & 0 & 0 & 1\end{matrix} ] ( \begin{matrix}u \\ v \\ w \\ 1\end{matrix} )$$

[symbol]

rmation matrix was determined by capturing the four points (−10,−10,0), (−10,10,0), (10,10,0), (10,−10,0) in the plane template, which is aligned to the O-xy plane in the machine coordinate.
Extract the Coordinate Transform Matrix Based on the Corresponding Points

Once we have a set of local landmark points on both surfaces of 3D images to be integrated, a 4×4 homogenous spatial transformation is derived to align them into a common coordinate system. For example, a least-square minimization method may be used to obtain the transformation.

This step includes denoting the corresponding fiducial point pairs on surface A and surface B as Aᵢ and Bᵢ, i=1,2, . . . , n. This allows the user to find a rigid transformation that minimizes the least-squared distance between the point pairs Aᵢ and Bᵢ. The index of the least-squared distance may be defined as:

$$I = {\sum }_{i = 1}^{n} {\vert {A}_{i} - R ( {B}_{i} - {B}_{c} ) - t \vert}^{2}$$

where T is a translation vector, i.e., the distance between the centroid of the point Aᵢ and the centroid of the point Bᵢ. R is found by constructing a cross-covariance matrix between centroid-adjusted pairs of points.

Not all measured points have the same error bound. In fact, for a 3D camera that is based on the structured light principle, the confidence of a measured point on a mesh depends on the surface angle with respect to the light source and camera's line-of-sight. A weight factor may be specified, wᵢ, to be a dot product of the mesh normal N at point P and the vector L that points from P to the light source. Therefore, the minimization problem becomes a weighted least-squares minimum:

$$I = {\sum }_{i = 1}^{n} {w}_{i} {\vert {A}_{i} - R ( {B}_{i} - {B}_{c} ) - t \vert}^{2}$$

The solution to such a problem is well known.
Software Tools Allowing Operators to Interactively Visualize and Quantify 3D Positioning Errors

The exemplary position error correction described above is an iterative procedure. Accordingly, it may be desirable to provide an operator with user-friendly and intuitive software tools that allow the operator to make the necessary adjustments quickly and effectively. A visualization tool is provided that displays the positioning error in real-time in all six degrees of freedom directly related to the machine coordinate system according to the results of 3D image registration. The quantitative description of the positional error and graphitic illustration of the head and head support device displacement may provide an intuitive guidance to make corrections. FIG. 5 presents an illustration of the interface screen (600) that has 6 DOF motion and force indicator.

Imaging Process and System

A ceiling-mounted 3D surface imaging system and method of acquiring accurate 3D surface images is discussed herein. Computational methods are also provided to estimate the true delivered dose given variations in patient geometry and to adaptively adjust the treatment plan when the delivered dose differs significantly from the planned dose with the aid of the finite element breast model.

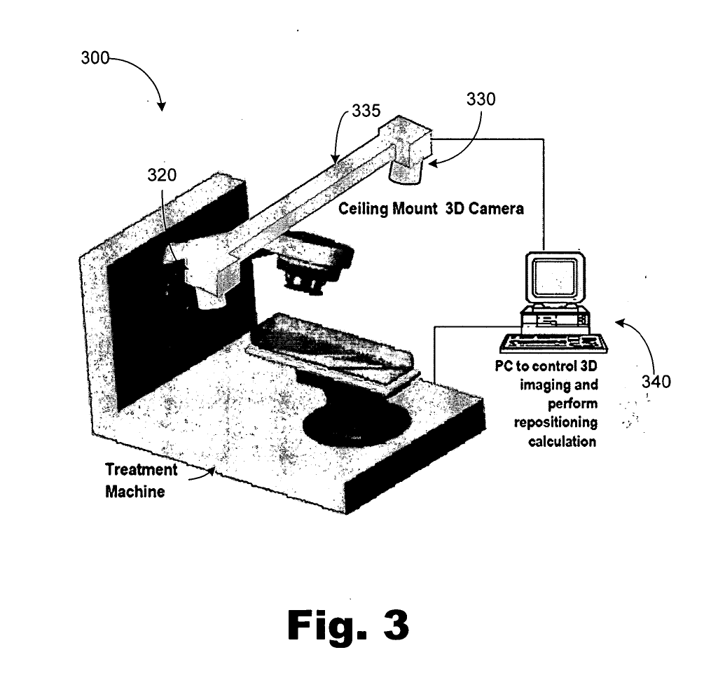

FIG. 3 illustrates a schematic view of a ceiling mounted 3D imaging system (300) for breast treatment. The stand-off distance between the 3D imaging system (300) and the object to be imaged (i.e., patient's breasts or head) may be approximately 2.35 meters. In order to achieve desired imaging accuracy (˜1 mm), the baseline between a rainbow projector (320) and an image sensor (330) may be extended. The image sensor may have a resolution of approximately 640×480 or higher. As a result, the sensor may have an accuracy of 500 microns or better. The components of the 3D camera, including the rainbow projector (320) and the image sensor (330) shown are mounted on a bar (335) to provide an appropriate convergence angle. The bar (335) is mounted on the ceiling of a treatment room, with cables connecting to a control host computer (340). According to other exemplary embodiments, the image sensor and rainbow projector (320) may be supported by a movable tripod system.

The problem of 3D image guided repositioning techniques for breast therapy treatment present a set of greater challenges: Due to the flexibility of breast, many related issues, such as gravity effect on 3D shape, effect of upper body and arm positions on the 3D shape of breasts, and volume changes during the period of treatment are all currently unknown or not well characterized, and are the state-of-the-art research topics representing significant technical challenges.

A ceiling mounted 3D camera system may be used to facilitate the fixed coordinate transformation between the 3D surface image system and the treatment machine. This fixed mounting may simplify the system calibration and repositioning calculation procedure, thus reducing the time required for repositioning the patient for each fractional treatment.

Surface Image Matching

As previously discussed, at the time of CT simulation, a reference surface scan is also made using the 3D camera. Because the 3D camera is calibrated with the CT isocenter, the relationship between the surface scan and internal structures can be found. Then, on each treatment fraction, the daily 3D surface scans will be matched with the reference surface scan to find the surface deformation present on each day. From the surface deformation, the displacement of surface nodes in the FEM model are computed and the deformation within the interior of the breast to locate the tumor is estimated.

Surface registration links two coordinate systems: reference (simulation) system and treatment system. It is accomplished in two stages: global matching and local matching. The best global match will compute the best affine transformation involving rotation, translation, scaling and shearing, while the best local match will be based on the energy minimization of a deformable surface. Matching is correspondence based, using linear combinations of both features and raw data readings in an iterative-closest-point style optimization. The features used may include, without limitation, surgical scars, nipples, and the bases of the breasts. Feature detection may be performed automatically based on 3D surface invariants computed for both the reference scan and the daily scan. The automatic feature detection may be assisted by user interaction in cases where the features are indistinct.

Visualization software for processing includes color-coded displays of surface match quality, feature match quality, and surface strain. The required control software may include feature selection and detection, correspondence selection, and model fitting.

A 3D surface imaging system will be discussed herein which makes use of finite-element deformation techniques for a variety of uses, including breast cancer radiotherapy. While the techniques will be discussed in the contact of breast cancer therapy, those of skill in the art will appreciate that the system and method may be used for any variety of applications, which include, without limitation, SRT. The 3D image of the breast surface may be acquired before each treatment fraction and morphed to match the reference surface image, as discussed above. Using the surface image as the boundary condition, the internal target volume is located by deforming the finite-element model of the breast. PBI treatment will be delivered after repositioning the patient. The residual error due to the rotation and deformation of the breast will be taken into account using accurate Monte Carlo dose calculations and adaptive treatment planning.

Unlike assuming a rigid correlation between surface landmarks and internal target volume as in current procedures, the internal tumor volume is derived with deformation using a finite element method. An adaptive treatment scheme, along with accurate dose prediction, may reduce or eliminate any residual errors and ensure the planned dose distribution is delivered at the end of the treatment course.

Such a system may provide the successful development of PBI possible, which in turn will offer opportunities of radiotherapy to a large number of BCT patients to improve the treatment outcome. Further, the system may make use of 3D surface imaging, finite element deformation, and adaptive inverse planning.

General Concept of Image Guided Adaptive Therapy for Partial Breast Irradiation

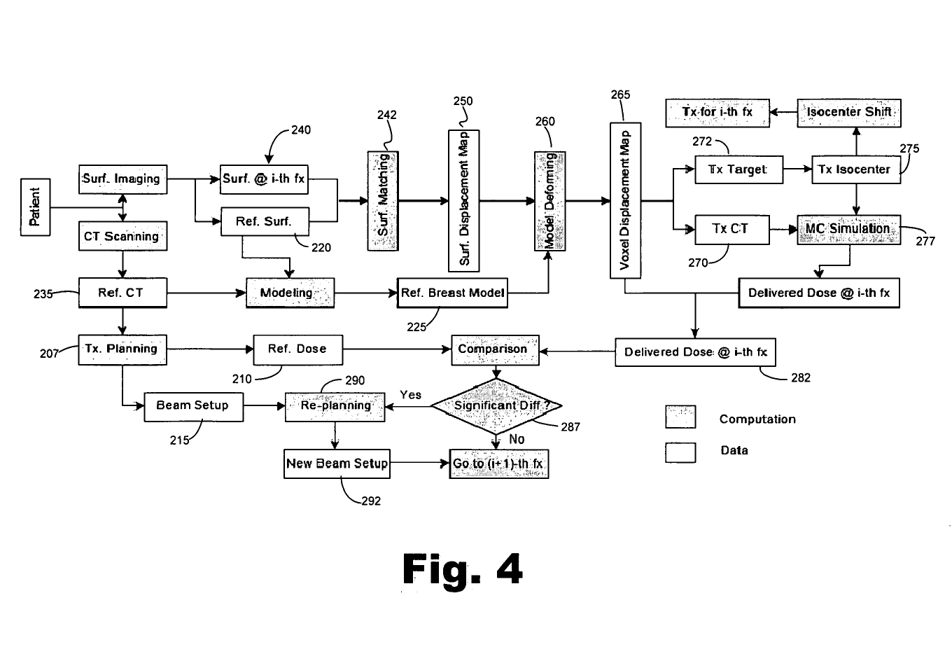

The flow chart of an image guided adaptive therapy, such as for partial breast irradiation (IGAT-PBI) process, is shown in FIG. 4. The process begins when the patient enters for treatment (200). A CT scan is acquired for treatment planning (Reference CT, 205). Photon beam IMRT may be combined with an electron beam for IGAT-PBI treatment. Treatment may be abbreviated as Tx, and will be used interchangeably with reference to FIG. 2. The treatment plan (207) includes the Reference Dose Distribution (210) and Beam Setup (215). The breast surface image is acquired using a 3D camera (Reference Surface) (220) at the time of CT scanning. A Reference Breast Model (225) is generated from the Reference Surface (220) and the Reference CT data (235) using a biomechanical finite-element model. At each treatment fraction, the patient will be initially setup using the conventional laser-skin marker technique, and then the 3D breast surface image (Measured Surface) (240) is taken using a 3D camera. The Measured Surface (240) is matched (242) with the Reference Surface (220) using deformable registration and a Surface Displacement Map (250) is generated. Using the Surface Displacement Map (250) as the boundary condition, the Reference Breast Model (225) is deformed (260), resulting in a Voxel Displacement Map (265). A set of new CT data (Treatment CT) (270) that represents patient geometry at the treatment time is calculated. The subsurface target location at the treatment time (Treatment Target) (272) is derived and thus the necessary isocenter shift is calculated. The treatment is then delivered with the shifted isocenter (Treatment Isocenter) (275). The dosimetric error caused by breast deformation may possibly not be eliminated by a simple isocenter shift, and therefore is estimated using a subsequent off-line Monte Carlo dose calculation (277). The calculation uses the updated patient geometry and shifted isocenter, and generates the Delivered Dose Distribution (280) from this fraction of treatment. Using the Voxel Displacement Maps (265), a Cumulative Dose Distribution (282) is generated. The Cumulative Dose Distribution (282) is then compared with the Reference Dose Distribution (210). If the difference is found to be clinically significant (287), the plan is re-optimized (290), which may include a new beam setup (292) in order to deliver a dose distribution as close to the Reference Dose Distribution (290) as possible at the end of treatment course.

3D Deformable Breast Model Based on Finite Element Techniques

Biomechanical models constructed using finite element techniques can be used to model the interrelation between different types of tissue by applying displacement or forces. The common steps for a calculation based on the finite element methods include pre-processing, solution, and post-processing. In the pre-processing step, property of the material is set and the finite element mesh is generated. In the solution step, the boundary conditions are applied to the finite element mesh. With respect to the mesh generation, boundary conditions used, and the assumed tissue properties, several different biomechanical breast modeling techniques are available. The use of finite element techniques will be discussed with reference to: (a) 3D breast mesh generation from CT data and surface images, (b) breast material property modeling, and (c) breast deformation modeling.

3D Mesh and Finite Element Model

Precision simulation of the human breasts deformation may make use of a high-fidelity biomechanical finite element breast model. For example, a tetrahedral mesh that fills the entire volume of the breast may be generated from the surface model. The property of the 3D mesh (finite elements) is registered with the volumetric images from CT scanners acquired during simulation and planning, therefore providing reliable knowledge of internal tissue distribution and tumor location based on the correspondence between the 3D surface image and the CT scans.

During the treatment session, a new 3D surface image is acquired and due to the high mobility and flexibility of breast, this new surface image may be quite different from the original reference 3D surface image acquired in the simulation session. The new 3D surface image provides a new set of boundary conditions to the deformable model. The finite element breast model will be deformed to comply with the new boundary condition. This deformable model therefore provides an effective and accurate means to locate the tumor for the deformed breast during treatment.

The process of generating finite element models using 3D surface images begins with acquiring 3D surface images of the chest. Thereafter, the 3D surface images of breasts are cut as areas of interest. Some pre-processing is performed on the 3D surface images of breasts to generate solid models of breasts. Some part of the pre-processing includes, without limitation, repairing the image, such as filling holes, removing degenerate parts, etc. After we obtain a 3D model, Delaunay triangulation algorithm and Delaunay refined algorithm are then used to produce finite element meshes on the solid models.

The resulting 3D meshed solid model is a geometric model of a human breast. Thereafter each node in the entire volume of the geometric model is assigned material properties in order to simulate the deformation behavior of the breast. The soft tissues of the human body consist of three elements: the epidermis, the dermis, and the subcutis from the anatomy point of view. These three elements can be simulated accurately by a layered structure of finite element models. However, for the fatty parts of the body like female breasts, which are full of subcutaneous (fat), a single layer is not enough to represent the subcutis layer. A volume mesh is used to represent the subcutis layer and specific consideration occurs on the tumor tissues.

3D surface image alone may not provide such volumetric information. Accordingly, the volumetric image from CT scans is registered with the 3D finite element model produced by the 3D surface image. In this way, the material properties of each element in the deformable model are known, based on CT information. This deformable model serves as the base for the patient-specific breast deformation during the treatment session.

Modeling Non-Linear Material Properties

The actual breast is composed of fat, glands with the capacity for milk production when stimulated by special hormones, blood vessels, milk ducts to transfer the milk from the glands to the nipples, and sensory nerves that give sensation to the breast. Assuming that tissues of all kinds can be modeled as isotropic and homogeneous. Most biological tissues display both a viscous (velocity dependent) and elastic response.

With these assumptions, it is possible to define the mechanical behavior of breast tissue using a single elastic modulus Eₙ, which is a function of strain εₙ, for tissue type n (σₙ is the stress) may be modeled mathematically according to Equation 1:

$${E}_{n} = \frac{\partial {σ}_{n}}{\partial {ɛ}_{n}} = f ( {ɛ}_{n} ) \qquad ( 1 )$$

Equation 1 is also known as Young's modulus, one of elastic constants needed to characterize the elastic behavior of a material. Eₙ does not change substantially for all stress and strain rates in a linear material model. Published values of the elastic modulus of component tissue of the breast vary by up to an order of magnitude, presumably due to the method of measurement or estimation. From a non-linear model E_{fat}=0.5197·ε²+0.0024·ε+0.0049, and E_{gland}=123.8889·ε³−11.7667·ε+0.012. The skin will be modeled as linear tissue with Young's modulus of 10 kPa and a thickness of approximately 1 mm.

Breast Model Volumetric Deformation Dynamics

Breast model deformation can be achieved by analyzing the mechanical response of each element inside the finite element model. The relation linking displacement and force is:
F=KU
where F is the force vector, K is the stiffness matrix, and U is the displacement of each node. If D is the material property matrix of each element:

$$[ D ] = \frac{E}{( 1 + v ) ( 1 - 2 v )} [ \begin{matrix}1 - v & v & v & 0 & 0 & 0 \\ v & 1 - v & v & 0 & 0 & 0 \\ v & v & 1 - v & 0 & 0 & 0 \\ 0 & 0 & 0 & \frac{1 - 2 v}{2} & 0 & 0 \\ 0 & 0 & 0 & 0 & \frac{1 - 2 v}{2} & 0 \\ 0 & 0 & 0 & 0 & 0 & \frac{1 - 2 v}{2}\end{matrix} ]$$

where E is the young's modulus and v is the Poisson's ratio. The stiffness matrix for each element may be expressed as:

$$K = {\int }_{V}^{} {B}^{T} DB ⅆ V$$

where B is the matrix relating strain to displacement, and V is the volume of the element. Force vector F can be expressed as:

$$F = {\int }_{V}^{} {B}^{T} σ ⅆ V$$

where a is the stress of the material.

Once all the element stiffness matrices and force vectors have been obtained, they are combined into a structure matrix equation (in the form of F=KU). This equation relates nodal displacements for the entire structure to nodal load.

If Ω_{N} is set of all node positions and Ω_{S} is the subset of all surface positions, then:
T_{N}:Ω_{N}→ [symbol] ³:(x,y,z) [symbol] (\tilde{x},\tilde{y},\tilde{z})
is the transformation of the breast model during deformation at the node positions. All surface nodes of the FEM model are constrained to the corresponding displacement vectors obtained from the 3D non-rigid registration T_{R}. For example:
T_{N}(x,y,z)=T_{R}(x,y,z) if (x,y,z)εΩS

After applying surface displacement as boundary conditions, the structure matrix equation can be solved to obtain unknown nodal displacement, i.e. the volume displacement. Assuming the FEM model relaxes to its lowest energy solution, direct elimination method may be adopted to solve the simultaneous F=KU for the consideration of robustness.

Monte Carlo Dose Calculation

Accurate dose calculation may be desirable for precision PBI. Monte Carlo simulation has been accepted as the most accurate dose predicting tool. The EGS4 Monte Carlo code MCSIM will be used. MCSIM is a variant of MCDOSE, which was originally developed at Stanford University specifically for radiotherapy treatment planning and treatment verification. The code can be used to perform dose calculation for both conventional photon/electron treatment, as well as IMRT, and has been well-benchmarked. See Ayyangar et al 1998,Jiang et al 1998,Rustgi et al 1998,Ma et al 1999,Deng et al 2000,Jiang et al 2000,Lee et al 2000,Li et al 2000,Ma et al 2000,Deng et al 2001,Jiang et al 2001,Sempau et al 2001.

The MCSIM code has been installed at MGH and used for the investigation of organ motion effect, and for WBI dose calculation. Several photon/electron beams at MGH have been commissioned and modeled for Monte Carlo simulation. Using the patient CT geometry at the current treatment fraction and the shifted isocenter, the delivered fractional dose distribution can be calculated using MCSIM. With the help of the voxel displacement maps, which give the correspondence of the voxels, the delivered fractional dose distributions can be added together. This will generate a delivered cumulative dose distribution. The Monte Carlo dose calculation and addition will be performed off-line after the fractional treatment. A user interface written in IDL (Interactive Data Language) may be used to facilitate the calculation with minimal human intervention.

It is estimated that a typical PBI dose calculation (one electron beam plus 3-5 photon beams) may take about two hours using a 2 GHz Pentium CPU. Multiple computer clusters may be used to speed up the computation. Currently, one computer cluster at NGH using 40 CPU with Condor clustering software, can be used simultaneously for Monte Carlo simulation.

Adaptive Inverse Planning

A few (<5) IMRT fields may combine with an electron field to deliver a conformal dose distribution for PBI treatment. IMRT optimization software may be used for inverse planning. This software has been successfully used for Monte Carlo based photon and electron IMRT optimization.

The delivered cumulative dose distribution is then compared with the reference dose distribution. When the dose difference is significant, the plan will be adjusted for remaining fractions, and the weights for the IMRT beamlets and electron fields will be re-optimized using our optimization software, taking into account the dose already delivered to each voxel.

In terms of the optimal time for plan adjustment, apparently, if the plan is changed too early, the errors that appear later may require further adjustment. On the other hand, if adjustment is delayed until the end of the course, there may not be enough fractions to compensate for the errors accumulated from previous fractions. Therefore, the optimal time may be around the middle point of the treatment course.

Biomechanical Deformable Finite-Element Breast Model

As previously introduced, a finite-element-based biomechanical breast model may be used to simulate the deformation of natural human breast. The 3D surface images are first processed to generate 3D solid models that are suitable to generate finite-element mesh. A 3D solid model is a solid bounded by a set of triangles such that two, and only two, triangles meet at an edge, and it is possible to traverse the solid by crossing the edges and moving from one face to the other. The relationship between vertices (V), edges (E), and faces (F) of a solid is:
V−E+F=2
which is known as Euler's Formula. Tumors are precisely located via the aid of CT scans after the generation of finite-element mesh. CT scans are also used to assign material properties to each node of the finite-element mesh.

Once the biomechanical deformable model of the breast is established using the CT scan data, the correct correspondence between the surface features and internal organ and tumor locations is obtained. The 3D surface images acquired during the treatment are used to define the boundary conditions of the deformation, and the software will alter the shape of the deformable model to fit the geometric constraints defined by the 3D surface image. The result of the deformation is a 3D breast model with current shape of breast and location of tumor. This deformed breast model will be used in the repositioning operation.

Overall System Configuration Design

The system discussed herein may be well adapted for several applications, including SRT applications and breast cancer treatment. As previously discussed, a ceiling mounted camera system may be used to acquire three dimensional images. Some aspects of one exemplary system will now be discussed in more detail. As previously discussed, the standoff distance between the 3D camera and the patient's face is approximately 2.35 meters in the ceiling mounted camera configuration, to achieve required imaging accuracy (˜1 mm). At this distance, the baseline distance between the rainbow projector and the imaging sensor is extended. The mechanical, electrical, and optical designs of each component are selected to comply with the convention of clinically deployable devices. Further, several design and installation rules may be provided to minimize the radiation effect on the 3D camera components.

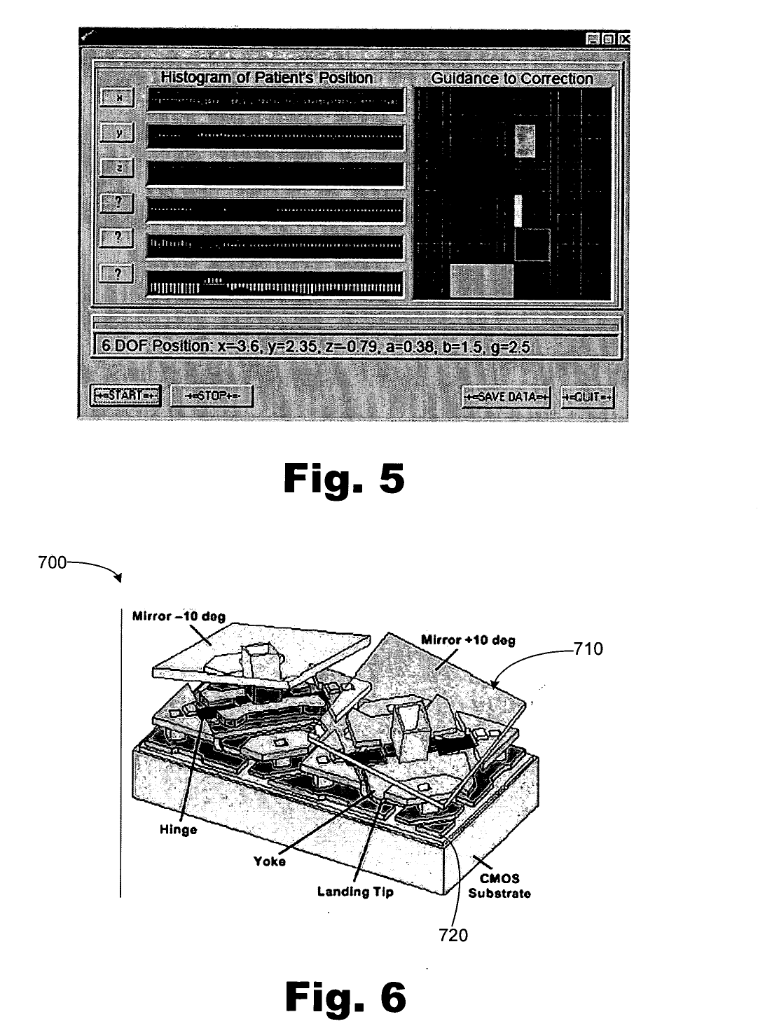

The rainbow light projector (320) shown makes use of reflective spatial light modulators, such as a Digital Micromirror Device (DMD) (700). The DMD, developed by Texas Instruments, is an array of fast switching digital micromirrors, monolithically integrated onto and controlled by a memory chip. As shown in FIG. 6, each digital light switch of the DMD includes an aluminum micromirror (710) with a dimension of approximately 13.7 μm square, which can reflect light in one of two directions depending on the state of an underlying memory cell. The mirror rotation is limited by mechanical stops (720) to ±10°. With the memory cell in the on state, the mirror rotates to +10°. With the memory cell in the off state, the mirror rotates to −10°. DMD architectures have a mechanical switching time of ˜15 μs and an optical switching time of ˜2 μs. The switching time of the mirrors is so fast that gray scale in images can be achieved through pulse width modulation (PWM) of the on and off (or “1” and “0”) time of each mirror according to a time line.

Unlike conventional light projectors where the illumination and projection optics can have a common optical axis, the optical axes of the illumination and projection optics for DMDs must have an angle determined by the DMD, which in the exemplary system discussed is approximately 24°.

The rainbow light projector (330) is shown schematically in FIG. 7. The rainbow light projector (320) includes illumination optics (800), which includes a lamp (805), such as a UHP lamp, a light integrator (810), condenser lens (820), two folding mirrors (830-1, 830-2), and a common UV filter lens (840) shared with projection optics (850). Lights from the UHP lamp are first collected by the light integrator (810), which is a tube with reflective inner sides formed by four mirrors. After multiple reflections, the light distribution at the exit of the light integrator (810) is almost uniform. Then the condenser lens (820) controls the shape and size of the light beam. To reduce the overall size, two folding mirrors (830-1, 830-2) are placed in the optical path. Mirror 1 (830-1) is a simple plane mirror, mirror 2 (830-2) is a non-spherical concave mirror to further reduce the optical path and improve uniformity of the light distribution. Just in front of the DMD chip (860), which includes an array of individual DMDs (700; FIG. 6), is placed a UV filtering lens (840) that is used to fend off UV light. The UV filtering lens (840) is also shared by the projection optics.

Re-Calibration and Quality Control Procedure

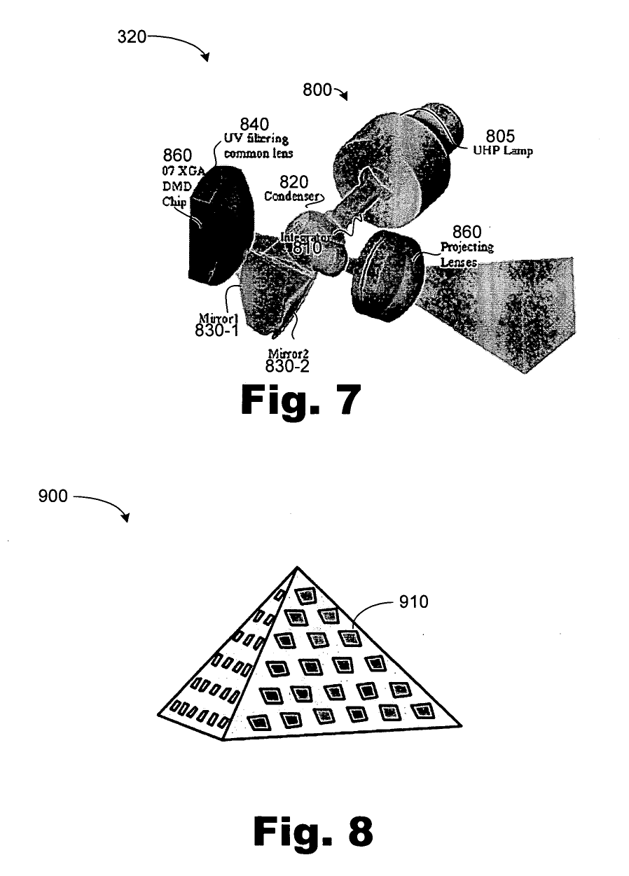

The ceiling mounted 3D camera needs may be periodically calibrated for quality control purposes. A calibration fixture (900) is shown in FIG. 8. The dimension of the fixture is known and the 3D locations of the features, such as corners of each square (910) painted on the pyramid surfaces, are known precisely. By placing the calibration fixture at a fixed position on a treatment couch and re-calibrating the camera parameters, the 3D coordinate relationship between camera and gantry system may then be re-established. The camera calibration procedure is straightforward and the algorithm is well-studied and proven. See “A Versatile Camera Calibration Technique for High-Accuracy 3D Machine Vision Metrology Using Off-the-Shelf TV Cameras and Lenses”, Roger Y. Tsai, IEEE J Robotics and Automation, Vol. RA-3, No. 4, 8/7, p 323, which is hereby incorporated by reference in its entirety.

Potential Commercial Applications

In addition to providing imaging and treatment planning for the head/neck/surface and/or breasts, the 3D imaging technology and software are also applicable to many other branches of medical fields. For example, 3D imaging techniques can be used for plastic and reconstructive surgery to provide quantitative measurement of the 3D shape of the human body for surgery planning, prediction, training, and education. 3D cameras can also be used to improve the fit of total contact burn masks. These burn masks' clear, rigid, and plastic form fit closely to the face, and are worn by patients who have received facial burns. Total contact burn masks provide evenly distributed pressure to compensate for the lack of tension in the burned tissue. The mask is worn continually throughout the healing process and acts to reduce the hypertrophic scarring. Other examples include the use in a prosthetics-orthotics (or other) computer-aided design and computer-aided manufacturing (CAD/CAM) system to compute a quantitative diagnostic measure of the patient's physiological state; as a measure of efficacy of a given medical treatment regimen; or added to an anthropometric/medical database. Anthropometrists can use our system to characterize the morphology of population. Forensic scientists can use the system to reconstruct facial dimensions from cranial materials.

The 3D imaging device can be used as a unique micro-imaging device to measure the internal body surfaces, such as 3D endoscope, blood vessel and colon scopes, 3D dental probe, etc. Beyond medical applications, the 3D video camera can be used in custom clothing industry, footwear product development, oxygen masks, and forensic analysis, etc. The apparel industry is interested in scanning customers to produce affordable, custom-tailored clothing. Garment makers might use the data to improve the fit of off-the-rack items, as well. Military can use 3D imaging techniques to improve the fit of uniforms, anti-G suits, and other equipment, and to redesign the layout of aircraft cockpits and crew stations.

The preceding description has been presented only to illustrate and describe the present method and apparatus. It is not intended to be exhaustive or to limit the disclosure to any precise form disclosed. Many modifications and variations are possible in light of the above teaching. It is intended that the scope of the disclosure be defined by the following claims.

1. A patient surface image guided therapy process comprising the steps of:
acquiring a three-dimensional reference image of an area to be treated,
acquiring a three-dimensional treatment image of said area to be treated;
matching said reference image to said treatment image; and
calculating any differences between said reference image and said treatment images to generate patient repositioning parameters.

2. The method of claim 1, and further comprising adjusting patient positioning or treatment machine configuration to achieve correct patient positioning.

3. The process of claim 1, wherein the step of capturing comprises the step of positioning a 3D Rainbow Camera above the treatment machine.

4. The process of claim 1, wherein the step of capturing comprises the step of operating a Rainbow 3D Camera for projecting a rainbow light pattern having a known spatially distributed structured light across the entire scene simultaneously.

5. The process of claim 1 wherein the step of preparing a pre-treatment CT scan further includes the additional step of calibrating the 3D Camera with the CT isocenter for identifying the relationship between the surface images and any internal structure or tumor.

6. The process of claim 1, including the additional step of modeling the inter-relationship between different types of tissue by applying displacement forces.

7. The process of claim 1, and further comprising an iterative fine alignment optimization process that includes searching for closest corresponding points between two images, optimizing via parameter perturbation, and determining whether a difference between a location of said positions is below a predetermined threshold.

8. The process of claim 1, wherein acquiring said three-dimensional reference image of said area to be treated comprises acquiring an image of a head and neck area.

9. The process of claim 1, wherein matching said reference image to said treatment image comprises selecting salient features.

10. The process of claim 9, wherein selecting said salient features comprises receiving an operator selection of said salient features.

11. The process of claim 10, wherein receiving said operator selection comprises processing a mouse click.

12. The process of claim 9, and further comprising performing an iterative fine alignment optimization process.

13. The process of claim 12, and further comprising performing an iterative closest point algorithm.

14. The process of claim 1, wherein acquiring said reference scan comprises acquiring a three-dimensional surface image.

15. The process of claim 1, wherein acquiring said reference scan comprises acquiring a CT scan, acquiring a three-dimensional surface image, and matching said CT scan to said three-dimensional surface image.

16. The method of claim 1, and further comprising performing deformable modeling operation on said area of interest.

17. The method of claim 16, wherein performing said deformable modeling operation includes using volumetric information from said reference image to generate a finite element model.

18. The method of claim 17, wherein generating said finite element model includes a plurality of layers having different material properties.

19. The method of claim 18, wherein thicknesses of said material properties are estimated using said reference image.

20. The method of claim 19, and further comprising matching surface boundary conditions of said reference image and said treatment image and estimating material properties of said area of interest based on said finite element model.

21. A system for surface image guided therapy, comprising:
a three-dimensional camera coupled to a processor, wherein said system is configured to acquire a three-dimensional reference image of an area to be treated,
acquire a three-dimensional treatment image of said area to be treated;
match said reference image to said treatment image; and
calculate any differences between said reference image and said treatment images to generate patient repositioning parameters.

22. The system of claim 16, wherein said three-dimensional camera includes light projector configured to project light of spatially varying wavelengths.

23. The system of claim 22, wherein said light projector comprises an array of digital micro-mirror devices.

24. The system of claim 21, wherein said three-dimensional camera is configured to be mounted above a treatment apparatus.

25. The system of claim 21, wherein said reference scan comprises a CT scan and a three-dimensional surface image.

26. The system of claim 21, wherein said processor is configured to plan a treatment based on said reference scan.

27. The system of claim 21, wherein said matching said reference image to said treatment image includes identifying salient features of said area to be treated.

28. The system of claim 27, wherein identifying said salient features includes receiving an operator selection of said salient features.

29. The system of claim 27, wherein said processor is further configured to perform an iterative closest point algorithm to match said images.

30. The system of claim 21, wherein said processor is configured to provide information related to adjustments relative to six degrees of freedom.

31. The system of claim 21, wherein said processor is configured to perform finite element analyses of said area to be treated.

32. The system of claim 21, wherein said finite element analysis includes an analysis of multiple mesh layers having different mechanical properties.

33. The system of claim 21, wherein said processor is configured to estimate a location of a target area within said area to be treated from said three-dimensional surface image.

34. The system of claim 21, wherein matching said reference scan to said three-dimensional surface image includes establishing a first fiducial point on said reference scan, searching for a corresponding point on said three-dimensional surface image, establishing a spatial relationship between said-fiducial point and said corresponding point to determine possible locations of other corresponding points on said three-dimensional surface scan; comparing feature vectors of corresponding points on said reference image and said three-dimensional surface scan find a rigid 4×4 homogenous transformation to minimize the weighted least-squared distance between pairs of points.

35. A system for imaging an area to be treated, comprising:
means for capturing a reference image;
means for capturing a treatment image;
means for registering said reference image and said treatment means; and
means for calculating a difference between said reference image and said treatment image.

36. The system of claim 35, and further comprising means for providing re-positioning information.

37. The system of claim 35, where said means for capturing a treatment image comprise means for capturing a three-dimensional surface image.

38. The system of claim 35, and further comprising means for positioning a patient.

39. The system of claim 35, and further comprising means for detecting positioning error.

40. The system of claim 39, wherein said means for detecting error positioning error comprises means for detecting positioning error of a portion of a patient's head and neck area.
